(19) United States
(12) Patent Application Publication
(10) Pub. No.: US 2026/0144761 A1
(43) Pub. Date: May 28, 2026

(54) TOPICAL PAIN PATCH

(71) Applicant: KT Health, LLC, American Fork, UT (US)

(72) Inventors: Howard Wu, Sandy, UT (US); Kade Applegate, Bluffdale, UT (US); Jessica Klodnicki, Park City, UT (US); Jeremy Newns, Orem, UT (US); Zach Decker, Park City, UT (US)

(21) Appl. No.: 18/956,595

(22) Filed: Nov. 22, 2024

(51) Int. Cl. A61K 9/70; A61K 31/045; A61K 47/32; A61K 47/34

(52) U.S. Cl. CPC A61K 9/7084; A61K 31/045; A61K 47/32; A61K 47/34

(57) ABSTRACT

Disclosed are systems, devices, and/or methods of use thereof regarding topical patches for providing pain relief to a user over a period of time, such as all-day or up to 8 hours. In various aspects, a topical patch for providing pain relief includes a cover layer, an adhesive layer disposed on the cover layer, and a hydrogel layer disposed on the adhesive layer. The adhesive layer may be for adhering the topical patch to skin of a user. The hydrogel layer may include a therapeutic component for providing pain relief to the user. The topical patch may be worn by the user for a period of up to around eight (8) hours.

### TECHNICAL FIELD

This disclosure relates generally to topical patches for providing pain relief to a user over a period of time. The topical patch may stay adhered to the body for up to 8 hours, even while the user engages in strenuous activity.

### SUMMARY

Disclosed are systems, devices, and/or methods of use thereof regarding topical patches for providing pain relief to a user over a period of time, such as all-day or up to 8 hours. In various aspects, a topical patch for providing pain relief includes a cover layer, an adhesive layer disposed on the cover layer, and a hydrogel layer disposed on the adhesive layer. The adhesive layer may be for adhering the topical patch to skin of a user. The hydrogel layer may include a therapeutic component for providing pain relief to the user. The topical patch may be worn by the user for a period of up to eight (8) hours.

In various aspects, a topical patch for providing pain relief includes a cover layer (e.g., woven, non-woven, knit, etc.) having four-way stretch (i.e., stretches both lengthwise and crosswise) and an adhesive layer disposed on the woven layer. The cover layer may be breathable and able to absorb sweat from a user's body. The adhesive layer may be for adhering the topical patch to skin of a user over a period of at least 6 hours, up to 8 hours, or longer. The adhesive layer may comprise a sweat-resistant adhesive material. The topical patch may also include a hydrogel layer disposed on the adhesive layer, with the hydrogel layer having a therapeutic component for providing pain relief to the user.

In various aspects, a method of manufacturing a topical patch for pain relief includes applying an adhesive layer to a surface of a textile, the textile including a woven fabric having four-way stretch. The method may also include incorporating a therapeutic component into a hydrogel layer and applying the hydrogel layer about or within a center of the adhesive layer, with the therapeutic component for providing pain relief over a period of up to 8 hours.

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1 illustrates a perspective view of a topical patch;

FIG. 2A illustrates a bottom view of a topical patch of a first size and FIG. 2B illustrates a bottom view of a topical patch of a second size;

FIG. 3 illustrates a cross-sectional view of the topical patch of any one of FIGS. 1 through 2B;

FIG. 4 illustrates placement of the topical patch of any of FIGS. 1 through 3 applied a user; and

FIG. 5 is a flowchart of an example method of manufacturing the topical patch.

### DETAILED DESCRIPTION

Topical pain relief alleviates aches or pains through the skin. For example, creams containing pain relief agents can be applied over an area of the user that is in pain in order to provide relief. Such relief can be provided for joint pains, muscle pain, or other types of pain. One form of topical pain relief is a patch that can be applied to the skin and worn for a period of time.

Generally, these patches can be worn for short periods of time, such as less than 4 hours. This requires replacement of the patch if pain relief is desired for a longer period of time. Additionally, these patches are generally worn when sedentary, such as sitting at a desk. Often, these patches fall off of the user during more strenuous activity, such as walking. This is due to the generally weak nature of the adhesive, which is susceptible to sweat or other moisture. Once these patches fall off the user, either through increased activity or exposure to water, the patch must be replaced with a new patch as the adhesive is no longer strong enough to re-stick the patch to the user.

Additionally, adhesive products that can be worn for longer periods of time and can withstand physical activity generally do not provide therapeutic pain relief. For example, tapes worn by athletes that are meant to cover large body parts to prevent abrasion or turf burn during athletic activity (e.g., the large tapes will prevent skin from tearing when the body contacts turf) stay adhered to the large body parts due to the entire surface area of the tape contacting the skin of the body. These tapes are usually trimmed to a specific size to match the user's specific body part.

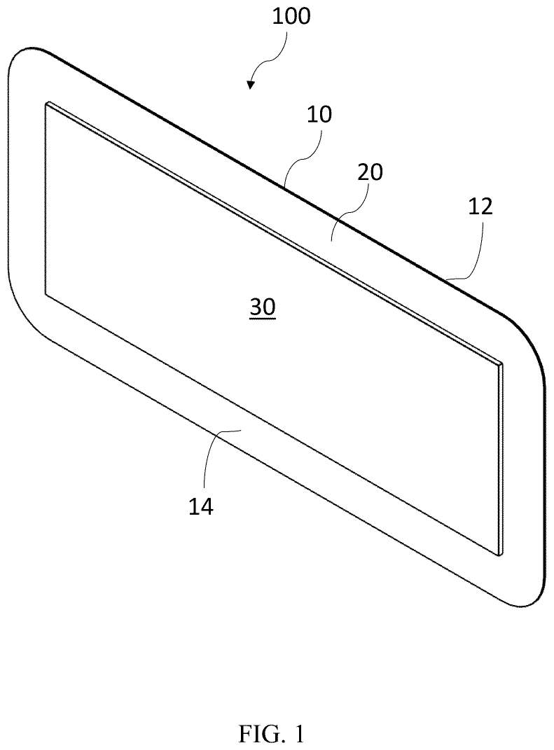

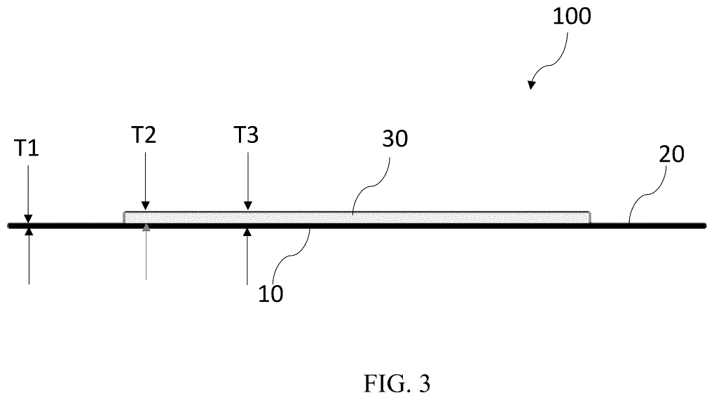

FIGS. 1 through 3 illustrate a topical patch 100 which can be worn for long periods of time (e.g., at least 6 hours, up to 8 hours, etc.) and is generally sweat-resistant, such that the patch 100 can also be worn during strenuous activity. That is, the topical patch 100 may adhere to the body for up to 8 hours. The topical patch 100 includes a cover layer 10, an adhesive layer 20 disposed on the cover layer 10, and a hydrogel layer 30 arranged and disposed on the adhesive layer 20.

The cover layer 10 may be a textile (e.g., woven or non-woven) layer or a fabric layer. For example, the cover layer 10 may include a polyester-spandex blend. Additionally, the cover layer 10 may have four-way stretch (i.e., stretches both lengthwise and crosswise), allowing the topical patch 100 to move with the user as the user moves. In some embodiments, the cover layer 10 may be an elastic four-way stretch film. By moving with the user, the topical patch 100 is less susceptible to peeling back at edges of the topical patch 100 when it is worn by a user. That is, movement by the topical patch 100 with the user maintains adherence of the topical patch 100 to the user for up to around eight (8) hours or more (e.g., 4, 5, 5.5, 6, 6.5, 7, 7.5, 8, 8.5, 9, 9.5, 10 hours, or a period of time within a range defined by any two of the foregoing values). In some embodiments, the topical patch 100 may be adhered to the user for at least 8 hours. This allows the user to engage in any type of movement at any level of activity or strain without concern that the topical patch 100 will peel off of the user.

The cover layer 10 may also be formed from a breathable, absorbent (e.g., moisture-wicking, etc.), and substantially abrasion-resistant material. This allows the topical patch 100 to quickly dry if it becomes wet, such as through perspiration or immersion into water. The abrasion-resistance means the topical patch 100 will not snag or catch on clothes when the topical patch 100 is worn under clothing. For example, the cover layer 10 of the topical patch 100 may enable clothing to glide over the topical patch 100, helping ensure that the topical patch 100 stays adhered to the user.

The adhesive layer 20 may be applied over an entirety (e.g., substantially an entirety) of the cover layer 10. For example, the adhesive layer 20 may be applied on an interior surface 14 of the cover layer 10, opposite from an exterior surface 12 of the cover layer 10. The adhesive layer 20 may be formed from an extra sticky adhesive that can withstand long wear times and is substantially water-and sweat-resistant). That is, the components of the adhesive layer 20 allow the user to engage in athletic and highly strenuous activity without concern that the patch 100 will fall off; the adhesive layer 20 maintains adherence of the topical patch 100 to the user for a period of up to 8 hours or more, even when the user engages in athletic activity for extended periods of time (e.g., 30 minutes or more, an hour or more, 90 minutes or more, two hours or more, etc.).

The adhesive layer 20 may be applied over the interior surface 14 of the cover layer 10 such that the adhesive material of the adhesive layer 20 is at least partially incorporated into the cover layer 10. The adhesive material may be substantially incorporated into a cover layer 10 having four-way stretch (e.g., sprayed onto fibers of the cover layer 10, etc.). The incorporation of the adhesive material into the cover layer 10 may enable the adhesive material and, thus, the adhesive layer 20 to move, or deform, with the cover layer 10 as the cover layer 10 moves, or deforms, with the user. Incorporation of the adhesive material into the cover layer may maintain adherence of the topical patch 100 to the user for up to around eight (8) hours or more (e.g., 4, 5, 5.5, 6, 6.5, 7, 7.5, 8, 8.5, 9, 9.5, 10 hours, etc.), allowing the user to engage in any type of movement at any level of strain without concern that the topical patch 100 will peel off of the user. In some embodiments, the topical patch 100 may stay adhered to the user for longer than 8 hours, such as 9, 10, 11, 12 hours, or a time period within a range defined by any two of the foregoing values.

The adhesive layer 20 may be incorporated into the cover layer 10, such as being incorporated into individual fibers that are woven or knit to create the cover layer 10. Additionally, and/or alternatively, the adhesive layer 20 may be applied to the interior surface 14 of the cover layer 10 (e.g., such as when the cover layer 10 is formed of a non-woven textile). The adhesive layer 20 may include polyacrylate and/or blends of polyacrylate. The adhesive layer 20 may be formed from a pressure-sensitive adhesive, such that the topical patch 100 can be applied and adhered to a user when pressure is applied. The adhesive layer 20 together with the cover layer 10 may have a thickness T1 of about 0.5 mm, such as 0.4, 0.45, 0.55 mm, or a thickness within a range defined by any two of the foregoing values. U.S. Pat. No. 8,216,425 titled “MANUFACTURE OF KINESIOLOGY TAPE” and issued on Jul. 10, 2012, the entire contents of which are herein incorporated by reference, describes methods for incorporating an adhesive, such as adhesive layer 20, into substrates, such as the cover layer 10.

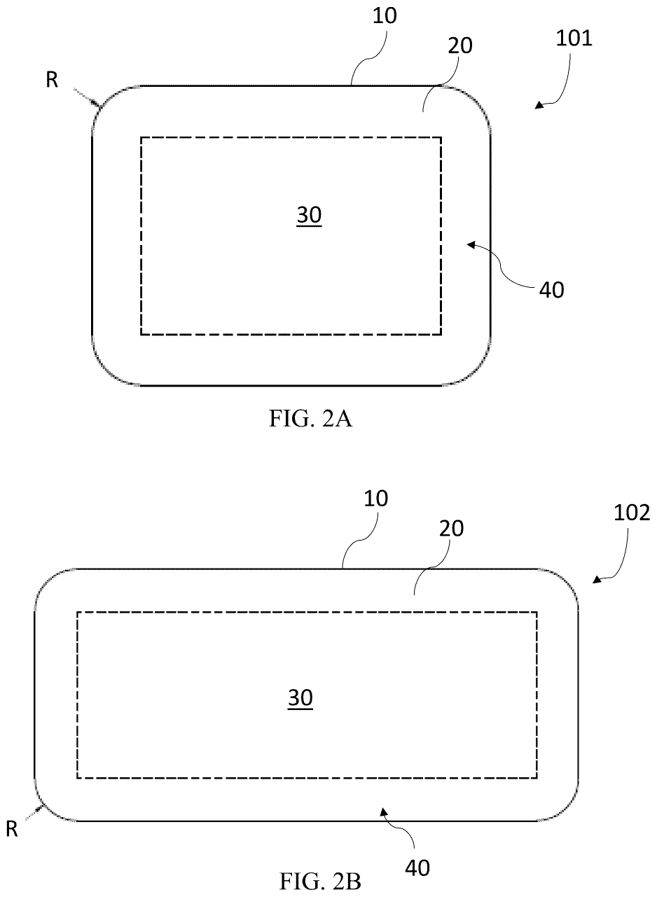

The hydrogel layer 30 may be disposed about a center of the topical patch 100. For example, referring to FIGS. 2A and 2B, the hydrogel layer 30 may be disposed on the adhesive layer 20 and the cover layer 10, such that a border 40 of adhesive laterally surrounds the hydrogel layer 30. This border 40 of adhesive securely adheres the topical patch 100 to a user. The hydrogel layer 30 may be disposed directly on the cover layer 10, with no intervening adhesive layer 20 between the hydrogel layer 30 and the cover layer 10. The border 40 of adhesive holds the assembled topical patch 100 to the user and ensures the topical patch 100 is spread uniformly across one side. The same adhesive that holds the assembled topical patch 100 to the user also holds the hydrogel layer 30 in place on the cover layer 10. Alternatively, the hydrogel layer 30 may be disposed on a portion of the adhesive layer 20, except for the border 40, such that the adhesive layer 20 both secures the hydrogel layer 30 in place on the cover layer 10 and adheres the topical patch 100 to the user.

In contrast to athletic tapes that adhere the entire surface area of the tape to the user, the border 40 of adhesive adheres the topical patch 100 to the user, allowing the hydrogel layer 30 to directly abut the skin of the user and provide topical relief. The border 40 of adhesive material may adequately secure and adhere the topical patch 100 to the user for up to 8 hours or longer without requiring the entire surface area of the topical patch 100 to be adhered to the user. The border 40 may have a width ranging from about 10 mm to about 18 mm, such as 11, 12, 12.5, 13, 13.5, 14, 14.5, 15, 15.5, 16, 16.5, 17, 17.5 mm, or a width within a range defined by any two of the foregoing values.

Though not illustrated, the topical patch 100 may include a backing that covers the hydrogel layer 30 and the border 40 of adhesive before the topical patch 100 has been applied to a user. Removal of the backing exposes the border 40 of adhesive, allowing the topical patch 100 to be applied and adhered to the user.

The hydrogel layer 30 is formed from water and natural and/or synthetic hydrophilic polymers. The hydrogel layer 30 also includes a medicinal therapeutic component that provides pain or other relief to the user while the topical patch 100 is worn. The therapeutic component may be menthol or blends of menthol with another appropriate component (e.g., eucalyptus, etc.). In some embodiments, the therapeutic component may include a medicine, such as a pain reliever. The therapeutic component may be included at a concentration (e.g., by weight) ranging from about 8% to about 14% by weight, such as 9%, 10%, 11%, 12%, 13%, or a concentration within a range defined by any two of the foregoing values. The hydrogel layer 30 may be configured in a manner known in the art to allow dissipation of the therapeutic component to the user over a period of up to around 8 hours or longer. For example, the hydrophilic polymers of the hydrogel layer 30 may create a matrix that captures or holds the therapeutic component and gradually releases the medicinal component. The gradual release allows the topical patch 100 to provide relief to the user during all-day wear of the topical patch 100.

The hydrogel layer 30 may have a thickness T2 of about 1.2 mm, such as 1, 1.27, 1.3, 1.4, 1.5 mm, or a thickness within a range defined by any two of the foregoing values. The hydrogel layer 30 together with the cover layer 10 and the adhesive layer 20 may have a thickness T3 of about 1.7 mm, such as 1.5, 1.6, 1.77, 1.8 mm, or a thickness within a range defined by any two of the foregoing values.

FIG. 2A illustrates a topical patch 100 of a first size 101. As illustrated, the first size 101 has a length of about 10.2 cm, a width of about 7.6 cm, and a thickness of about 0.18 cm. FIG. 2B illustrates a topical patch 100 of a second size 102. As illustrated, the second size 102 has a length of about 16.5 cm, a width of about 7.6 cm, and a thickness of about 0.18 cm. The hydrogel layer 30 may have a length ranging from 7.5 cm to about 14 cm and a height of about 5 cm. Corners of the topical patch 100 may have a radius of about 12 mm. The rounded corners prevent peeling of the topical patch 100 during wear by the user and during strenuous activity. The border 40 of adhesive may have a width of about 12 mm.

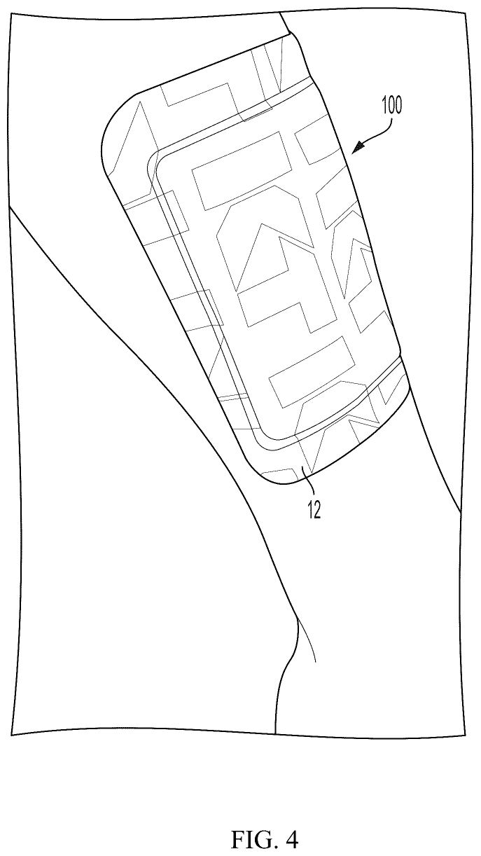

FIG. 4 illustrates the topical patch 100 of FIGS. 1 through 3 applied to a user. As illustrated, the topical patch 100 has been applied to an arm of the user. The topical patch 100 may be applied to almost any area of the user, such as a leg, a thigh, an upper arm, a lower arm, the calf, foot, back, neck, shoulder, knee, etc. The exterior surface 12 of the topical patch 100 may be exposed when the topical patch 100 is worn. As the topical patch 100 includes an abrasion-resistant cover layer 10, the exterior surface 12 may glide under any clothes (e.g., a shirt), without snagging or peeling up.

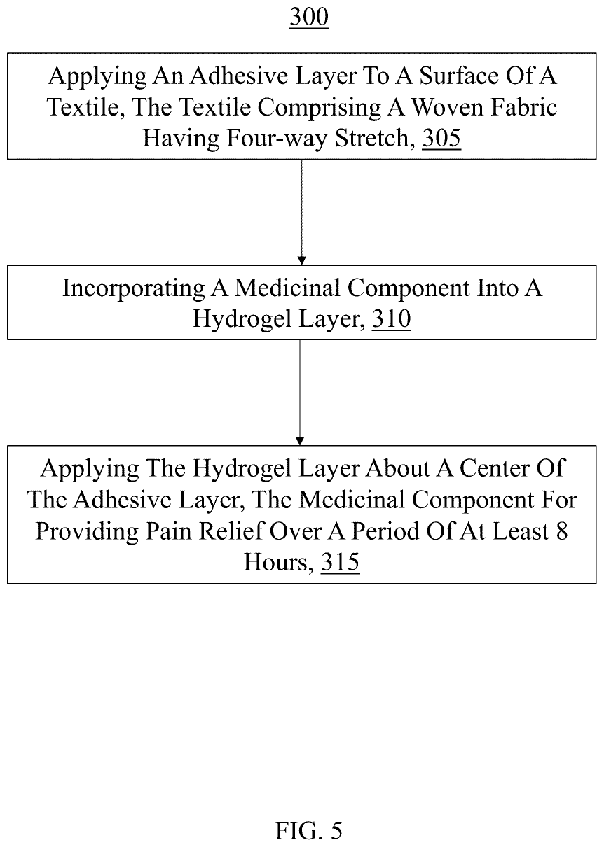

FIG. 5 is a flowchart of an example method 300 of manufacturing the topical patch, such as the topical patch 100 of FIGS. 1A through 3. The method 300 may include applying an adhesive layer to a surface of a textile, with the textile including a woven, non-woven, or knit fabric having four-way stretch, at 305. The method 300 may also include incorporating a therapeutic component into a hydrogel layer, at 310, and applying the hydrogel layer about a center of the adhesive layer, at 315. The therapeutic component may be for providing pain relief over a period of time. The adhesive layer may be substantially sweat-resistant, allowing the patch to be worn over the period of up to 8 hours without decreasing in adhesiveness.

### Additional Terms and Definitions

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination.

In one embodiment, the terms “about” and “approximately” refer to numerical parameters within 10% of the indicated range. The terms “a,” “an,” “the,” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the author(s) of this disclosure for carrying out the embodiments disclosed herein. Of course, variations on these described embodiments will become apparent to those of ordinary skill in the art upon reading the foregoing description. The author(s) expects skilled artisans to employ such variations as appropriate, and the author(s) intends for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of this disclosure so claimed are inherently or expressly described and enabled herein.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A topical patch for providing pain relief, the topical patch comprising:
a cover layer;
an adhesive layer disposed on the cover layer, the adhesive layer for adhering the topical patch to skin of a user; and
a hydrogel layer disposed on the adhesive layer, the hydrogel layer comprising a therapeutic component for providing pain relief to the user,
wherein the topical patch is adhered to the user for a period of at least 6 hours.

2. The topical patch of claim 1, wherein the adhesive layer comprises a pressure-sensitive adhesive component which forms a bond when pressure is applied.

3. The topical patch of claim 1, wherein the cover layer comprises a polyester-spandex blend having four-way stretch.

4. The topical patch of claim 3, wherein the four-way stretch allows the cover layer to move with the user, thereby maintaining adherence of the topical patch to the user for up to 8 hours.

5. The topical patch of claim 3, wherein the cover layer is substantially sweat-resistant, thereby maintaining adherence of the topical patch to the user for up to 8 hours.

6. The topical patch of claim 1, wherein the adhesive layer comprises polyacrylate.

7. The topical patch of claim 1, wherein the adhesive layer is sweat-resistant and maintains adherence of the topical patch to the user during athletic activity.

8. The topical patch of claim 1, wherein the hydrogel layer comprises natural and/or synthetic hydrophilic polymers.

9. The topical patch of claim 8, wherein the hydrogel layer allows diffusion of the therapeutic component to the user.

10. The topical patch of claim 1, wherein the therapeutic component comprises menthol.

11. The topical patch of claim 1, wherein the therapeutic component has a concentration ranging from about 8% to about 14% by weight of the hydrogel layer.

12. A topical patch for providing pain relief, the topical patch comprising:
a cover layer having four-way stretch;
an adhesive layer disposed on the cover layer, the adhesive layer for adhering the topical patch to skin of a user over a period of at least 6 hours; and
a hydrogel layer disposed on the adhesive layer, the hydrogel layer comprising a therapeutic component for providing pain relief to the user.

13. The topical patch of claim 12, wherein the hydrogel layer is positioned substantially within a center of the topical patch.

14. The topical patch of claim 12, wherein the hydrogel layer has a thickness of about 1.2 mm.

15. The topical patch of claim 12, wherein the topical patch has a length of about 16 cm, a width of about 7 cm, and a thickness of about 0.2 cm.

16. The topical patch of claim 12, wherein the topical patch has a length of about 10 cm, a width of about 7 cm, and a thickness of about 0.2 cm.

17. The topical patch of claim 12, wherein the cover layer is a woven layer being substantially abrasion-resistant, preventing snags or tears when the topical patch is positioned underneath clothes of the user.

18. A method of manufacturing a topical patch for pain relief, the method comprising:
applying an adhesive layer to a surface of a textile, the cover comprising a woven, non-woven, knit fabric, or film having four-way stretch, the adhesive layer for adhering the topical patch to a user for up to 8 hours;
incorporating a therapeutic component into a hydrogel layer; and
applying the hydrogel layer over a center of the adhesive layer.

19. The method of claim 18, wherein the adhesive layer is sweat-resistant and comprises polyacrylate.

20. The method of claim 18, wherein applying the hydrogel layer about a center of the adhesive layer creates a border of adhesive surrounding the hydrogel layer, the border of adhesive for adhering the topical patch to the user for up to 8 hours.
